(19) United States
(12) Patent Application Publication
(10) Pub. No.: US 2024/0335608 A1
(43) Pub. Date: Oct. 10, 2024

(54) CONTROL SAFEGUARDS FOR INFUSION PUMP

(71) Applicant: B. Braun Medical Inc., Bethlehem, PA (US)

(72) Inventors: Michael Robert Pavlovic, Stroudsburg, PA (US); Vladislav Los, Palmerton, PA (US)

(73) Assignee: B. Braun Medical Inc., Bethlehem, PA (US)

(21) Appl. No.: 18/131,460

(22) Filed: Apr. 6, 2023

(51) Int. Cl. A61M 5/172; A61M 5/142; A61M 5/168

(52) U.S. Cl. CPC A61M 5/172; A61M 5/14228; A61M 5/16813; A61M 2205/502

(57) ABSTRACT

Infusion pump control safeguards are provided by using multiple controller cores to ensure proper operation of an infusion pump. Each controller core controls specific operations of the pump and is interconnected with each other to monitor the operation of the other controller cores. The controller cores may include a pump controller core, the supervisor controller core, a System Controller Core, and a User Interface Controller Core. The Pump Controller Core is dedicated to control pump motor movement. No other controller core has direct access to the pump motor with a limited exception of the supervisor controller core, which can stop power flow to the pump motor in the event any one of the other controller cores is determined to be behaving improperly.

### FIELD OF THE INVENTION

The present disclosure is related to infusion pumps and, more particularly, to control safeguards whereby a collection of interconnected controller cores is used to control the infusion pump to ensure proper operation and to prevent possibility of overdose.

### BACKGROUND

Infusion pumps deliver controlled doses of fluids such as medications, analgesics, and nutrition to patients. Infusion pumps are particularly well suited to delivering controlled doses of fluids over long periods of time, e.g., several hours or days. While many infusion pumps are designed for bedside use, there are ambulatory versions available. Ambulatory infusion pumps allow a patient to move around while the infusion pump is in use.

Syringe pumps and peristaltic pumps are two conventional types of infusion pumps. A syringe pump depresses a cylinder within a syringe to deliver fluid from the syringe to a patient. A peristaltic pump acts on a tube to control the rate of fluid flow through the tube from a bottle or bag of fluid to a patient. Precise delivery of fluids is desirable to optimize treatment of a patient as there are many fluids where small variations can be critical.

Infusion pumps are typically set up and programmed by medical professionals in a medical setting. However, ambulatory infusion pumps may be operated by a patient in a home setting. Control safeguards for safe programming and operation of infusion pumps by untrained users is useful to avoid operator or system error.

### SUMMARY

Examples described herein are directed to methods and infusion pumps for delivering fluids to a patient. In sample configurations, infusion pump control safeguards are provided by using multiple controller cores to ensure proper operation of the infusion pump. The controller cores are interconnected to monitor the operation of each other.

In sample configurations, the controller cores include a Pump Controller Core, a Supervisor Controller Core, a System Controller Core, and a User Interface Controller Core. The Pump Controller Core is dedicated to control pump motor movement. The Pump Controller Core is responsible for controlling the pump motor movement (e.g., start/stop/rotation rate/etc.). No other controller core has direct access to the pump motor (with the limited exception of the Supervisor Controller Core). The Supervisor Controller Core is dedicated to pump supervisory operations. The Supervisor Controller Core can stop power flow to the pump motor in the event any one of the other controller cores is determined to be behaving improperly. The Supervisor Controller Core monitors the health of all other controller cores in the pump. In the event that a controller core is behaving improperly, the Supervisor Controller Core is capable of stopping power flow to the pump motor by means of switching off the power source to the motor. The remaining controller cores (System Controller Core and User Interface Controller Core) control other operations of the pump (e.g., graphical user input/output, infusion state logic, alarms, configurations, etc.). These controller cores are interconnected with each other, the Pump Controller Core, and the Supervisor Controller Core. Each controller core monitors the operation of the other controller cores and may raise alarms when improper behavior is detected. If a core is having a problem that is not detected by other controller cores, the Supervisor Controller Core may take action.

In sample configurations, an infusion pump is described that includes a pump motor configured to pump a fluid through a tube, a power source for the pump motor, and a controller for controlling the pump motor. The controller is adapted to include multiple controller cores that work together to ensure proper operation of the pump motor. Each controller core controls specific operations of the infusion pump and is interconnected with each other controller core to monitor operation of the other controller cores. The multiple controller cores are adapted to stop power flow to the pump motor from the power source in the event any one of the multiple controller cores is determined to be behaving improperly. The Supervisor Controller Core may monitor the health of all other controller cores in the infusion pump. In the sample configurations, the multiple controller cores include a Pump Controller Core that controls movement of the pump motor. The multiple controller cores also may include a System Controller Core and a User Interface Controller Core that control operations of the infusion pump including at least one of graphical user input/output, infusion state logic, alarms, or configurations.

The sample configurations further include a method of providing control safeguards for an infusion pump, a pump motor configured to pump a fluid through a tube, a power source for the pump motor, and a controller for controlling the pump motor where the controller includes multiple controller cores adapted to ensure proper operation of the pump motor, each controller core controlling specific operations of the infusion pump and being interconnected with each other controller core. The method includes each controller core monitoring operation of the other controller cores and stopping power flow to the pump motor from the power source in the event that any one of the multiple controller cores is determined to be behaving improperly. The method may further include the Supervisor Controller Core monitoring the health of all other controller cores of the multiple controller cores in the infusion pump and controlling movement of the pump motor using a Pump Controller Core of the multiple controller cores. Operations of the infusion pump including at least one of graphical user input/output, infusion state logic, alarms, or configurations may be controlled using at least one of a System Controller Core or a User Interface Controller Core of the multiple controller cores.

### BRIEF DESCRIPTION OF THE DRAWINGS

The drawing figures depict multiple views of one or more implementations, by way of example only, not by way of limitations. In the figures, like reference numerals refer to the same or similar elements. The same numeral is used to represent the same or similar element across the multiple views. If multiple elements of the same or similar type are present, a letter may be used to distinguish between the multiple elements. When the multiple elements are referred to collectively or a non-specific one of the multiple elements is being referenced, the letter designation may be dropped.

FIG. 1 is a perspective view of an example ambulatory peristaltic infusion pump.

FIG. 2 is a perspective view of an example cassette with a free flow prevention clamp for use with the ambulatory peristaltic infusion pump of FIG. 1.

FIG. 3 is a partial perspective view of the ambulatory peristaltic infusion pump of FIG. 1 illustrating cams that engage the free flow prevention clamp when the cassette is coupled to the ambulatory peristaltic infusion pump.

FIGS. 4 and 5 are cutaway views of the ambulatory peristaltic infusion pump of FIG. 1 illustrating pump sliders and cam rods for moving the pump sliders.

FIG. 6 is a diagram illustrating multiple controller cores to ensure proper operation of the ambulatory peristaltic infusion pump in a sample configuration.

FIG. 7 is a functional block diagram illustrating a general-purpose computer hardware platform configured to implement the functional examples described with respect to FIGS. 1-6.

FIG. 8 is another functional block diagram illustrating a general-purpose computer hardware platform configured to implement the functional examples described with respect to FIGS. 1-6.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well-known methods, procedures, components, and circuitry have been described at a relatively high-level, without detail, in order to avoid unnecessarily obscuring aspects of the present teachings. Moreover, while described with respect to an ambulatory peristaltic infusion pump for pain management, homecare, outpatient infusions, and the like, it will be appreciated by those skilled in the art that the pump safeguards described herein may be used with a variety of other pump types.

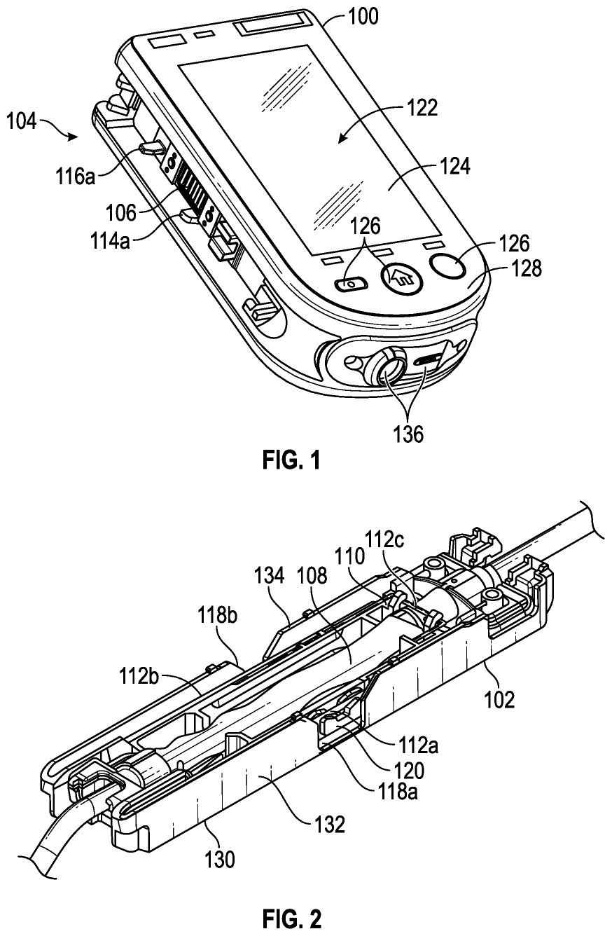

FIG. 1 depicts an example ambulatory peristaltic infusion pump 100, while FIG. 2 depicts an example cassette 102 for use with the ambulatory peristaltic infusion pump 100. The ambulatory peristaltic infusion pump 100 includes a receptacle 104 configured to receive the cassette 102. A peristaltic pump 106 within the receptacle 104 acts upon a tube 108 extending through a channel within the cassette 102 to pump fluid from a fluid container (e.g., a bag or a bottle; not shown) into a patient. An example free flow prevention clamp 110 is positioned within the cassette 102 to allow fluid flow through the tube 108 when the cassette 102 is coupled to the ambulatory peristaltic infusion pump 100 within the receptacle 104 (during which time the peristaltic pump 106 controls fluid flow through the tube 108) and to selectively cut off fluid flow through the tube 108 when the cassette 102 is not coupled to the ambulatory peristaltic infusion pump 100 in order to prevent unintentional fluid flow through the tube 108 (e.g., free flow).

The ambulatory peristaltic infusion pump 100 includes a user interface 122 for interacting with the ambulatory peristaltic infusion pump 100. The illustrated user interface 122 includes a display 124 (which may be a touchscreen) and buttons 126. A user controls the operation of the ambulatory peristaltic infusion pump 100 via the user interface 122 and via buttons 126, as needed. The ambulatory peristaltic infusion pump 100 additionally includes a housing 128 for containing and supporting the components of the ambulatory peristaltic infusion pump 100 such as the peristaltic pump 106, electronics, power supplies, and the like.

The free flow prevention clamp 110 includes a first elongate section 112a, a second elongate section 112b, and a clamping section 112c. The housing 130 of the cassette 102 supports the free flow prevention clamp 110. The clamping section 112c is positioned within the cassette geometry such that, when the cassette 102 is received within the receptacle 104 of the ambulatory peristaltic infusion pump 100, the clamping section 112c extends across the channel receiving the tube 108. The housing 130 of the cassette 102 may be rigid plastic or other material capable of supporting the tube 108 and free flow prevention clamp 110.

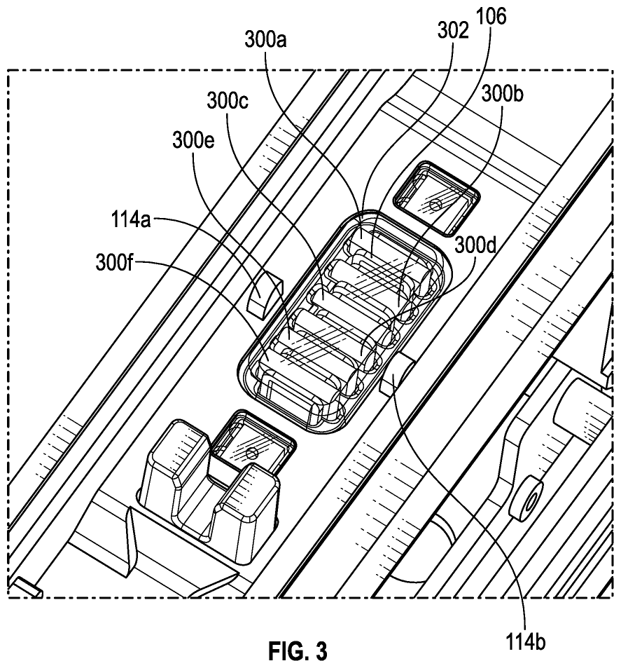

The ambulatory peristaltic infusion pump 100 also includes a pair of arc cams 114a and 114b (FIG. 3). First arc cam 114a is shown on one side of the receptacle illustrated in FIG. 1, but the second arc cam 114b is hidden from view. The pair of arc cams 114a and 114b engage the elongate sections 112a, 112b of the free flow prevention clamp 110 in order to lift the clamping section 112c. Additionally, the ambulatory pump 100 includes a pair of wedge cams 116a and 116b. A first wedge cam 116a is shown on one side of the receptacle 104 illustrated in FIG. 1, but the second wedge cam 116b is hidden from view. The pair of wedge cams 116a and 116b transition the free flow prevention clamp 110 from an open, manufactured/shipped state to an operational state, which is described in further detail below.

The cassette 102 also includes a first cutout 118a in a sidewall 132 of the cassette 102 and a second cutout 118b in an opposite sidewall 134 of the cassette 102. Additionally, the cassette 102 includes a touch pad 120 positioned on the first elongate section 112a adjacent a mid-point of the first elongate section 112a and the first cutout 118a. The touch pad 120 and cutout 118a together facilitate engagement of the first elongate section 112a by a finger of an operator in order to manually lift the clamping section 112c to allow fluid flow through the tube 108 (e.g., for priming the cassette 102) when the cassette 102 is not received within the receptacle 104 of the ambulatory peristaltic infusion pump 100. The touch pad 120 may be a press fit piece of rigid plastic. Although the touch pad 120 is illustrated as only on the first elongate section 112a, the touch pad 120 also may be provided on the second elongate section 112b.

The ambulatory infusion pump 100 further includes connector ports 136 that provide electronic access for control and for powering the ambulatory infusion pump 100 when used in the configurations described below.

FIG. 3 depicts the arc cams 114a and 114b and peristaltic pump 106 of the ambulatory peristaltic infusion pump 100. The peristaltic pump 106 includes multiple pump fingers 300 (six pump fingers 300a-f illustrated in FIG. 3). A flexible barrier (seal) 302 separates the pump fingers 300 (and other pump components of a pumping mechanism) from the receptacle 104 receiving the cassette 102 with the tube 108. The flexible barrier 302 provides a barrier between the fluid delivery apparatus/cassette and the pumping mechanism to prevent fluid from damaging components of the pumping mechanism.

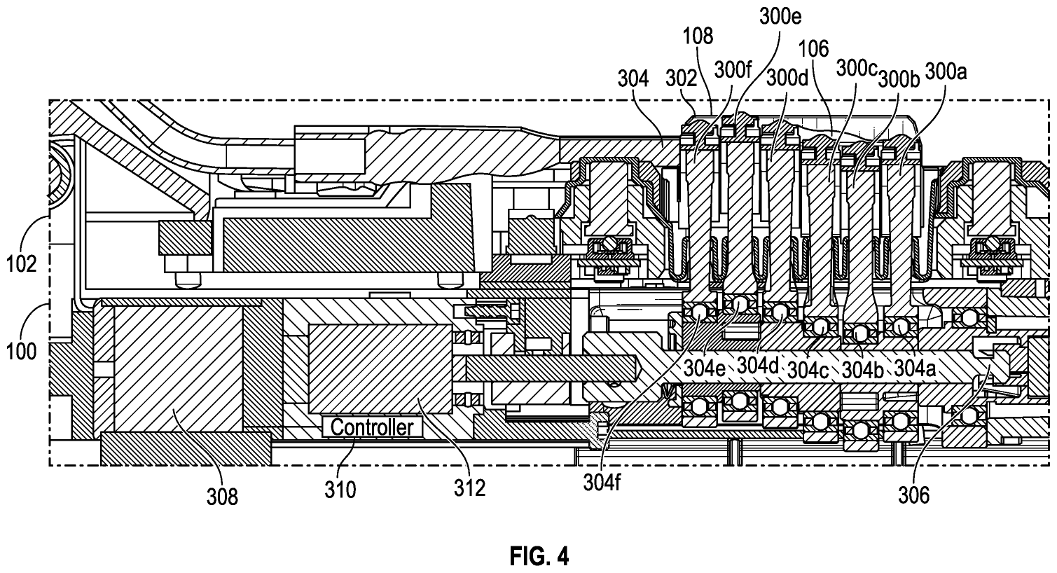

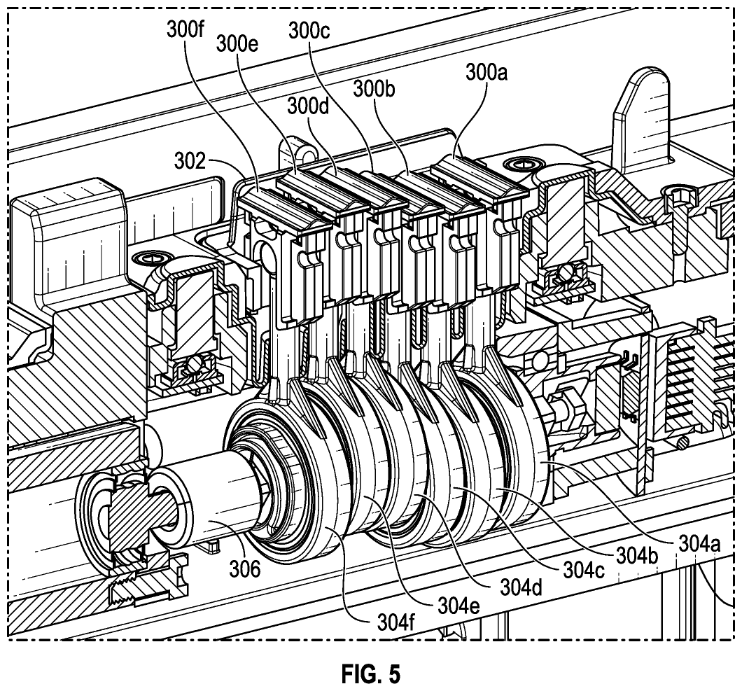

FIGS. 4 and 5 are cutaway views of the ambulatory peristaltic infusion pump 100 with the cassette 102 inserted into the receptacle 104 of the ambulatory infusion pump 100. Multiple cams 304 (six cams 304a-f) supported by a camshaft 306 act on respective pump sliders 300a-300f. The cams 304a-304f respectively raise and lower the pump sliders 300a-300f, which engage the tube 108 of the cassette 102 in order to force fluid though the tube 108. A pump motor 308 under control of a controller 310 turns the camshaft 306 by way of a gearbox 312. As the camshaft 306 turns, the cams 304a-300f, which are offset from each other in an axial direction, raise and lower respective pump sliders 300a-300f. For example, cam 304a raises and lowers pump slider 300a; cam 304b raises and lowers pump slider 300b, and the like. The controller 310 may be a standalone or embedded processor configured to carry out instructions in order to control operations of the ambulatory peristaltic infusion pump 100.

The pump controller 310 may include a User Interface Controller Core/System Controller Core such as a dual core 32 bit processor from NXP of Eindhoven, Netherlands (e.g., model #MCIMX7S5EVM08SC), a Pump Controller Core from NXP (e.g., model #MKV31F512VLH12), a Supervisor Controller Core from NXP (e.g., model #MKL17Z128VMP4), a pump motor driver from ST Microelectronics of Geneva, Switzerland (e.g., model #STSPIN250), and a magnetic encoder from Austriamicrosystems of Premstaetten, Austria (e.g., model number AS5601-ASOM). The microcontroller receives pump camshaft revolutions per minute (RPM) corresponding to the infusion rate from a system control core of the main processor. The microcontroller develops a pulse width modulation (PWM) motor drive parameter relating to the desired camshaft RPM. The PWM output of the microcontroller becomes the motor drive input to the pump motor driver, which contains motor drive transistors and protection circuitry. The rotation of the camshaft 306 of the pumping mechanism is measured by the magnetic encoder. At specified time intervals, the output of the encoder is read by the microcontroller, which uses the encoder value to compute the speed of the camshaft 306 and the position of the pump rotation. These values are then used to modify the PWM output to maintain the correct camshaft RPM.

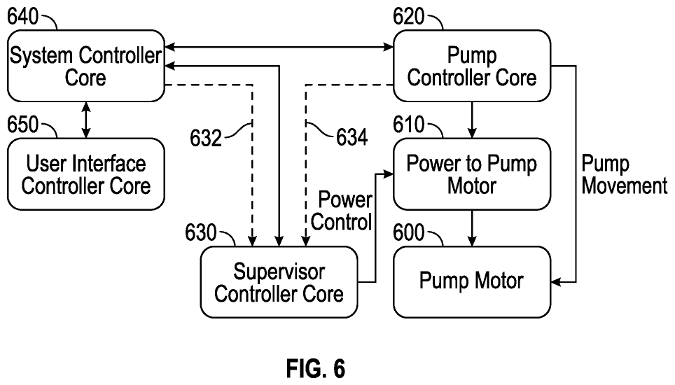

In sample configurations, the controller 310 includes multiple cores designed to ensure safe operation of the ambulatory peristaltic infusion pump 100. As described above with respect to FIGS. 1-5, the ambulatory peristaltic infusion pump 100 includes a peristaltic pump 106 configured to deliver fluid (e.g., pain medication) to a patient in response to controller 310. FIG. 6 is a diagram illustrating multiple controller cores used as controller 310 to ensure safe operation of the peristaltic pump 106 in sample configurations. As shown in FIG. 6, the peristaltic pump 106 includes a pump motor 600 that is powered by a power source 610 and controlled by a collection of interconnected controller cores 620-650 that monitor the operation of each other and process communications within the peristaltic pump 106. In sample configurations, the interconnected controller cores 620-650 monitor each other through use of a communication protocol whereby each controller core 620-650 sends out heartbeats that the other controller cores 620-650 “listen” for. If a controller core 620-650 goes offline because it stops sending heartbeats, the other controller cores 620-650 will know and raise a device alarm that completely stops pump operation.

In the configuration illustrated in FIG. 6, the Pump Controller Core 620 is dedicated to control movement of the pump motor 600. The Pump Controller Core 620 is responsible for controlling movement of the pump motor 600 (e.g., start/stop/rotation rate/etc.). No other controller core has direct access to the pump motor 600 (with the limited exception of the Supervisor Controller Core). The other controller cores may monitor the Pump Controller Core 620 to make sure motor movement is performed as expected. For example, when both configuring and running an infusion, there are safeguards performed by both the User Interface Controller Core 650 and the System Control Core 640 to verify that an infusion is being configured within the limits defined. When running an infusion, there are infusion status messages being transmitted that are used to verify proper operation. If parameters are detected as invalid, the appropriate alarms will be raised.

The Supervisor Controller Core 630 is dedicated to supervisory operations for the pump motor 600. The Supervisor Controller Core 630 can stop power flow to the pump motor 600 by controlling the power to the pump motor at power source 610 in the event any one of the other controller cores 620, 640, or 650 is determined to be behaving improperly. The Supervisor Controller Core 630 also may monitor the health of all other controller cores in the ambulatory peristaltic infusion pump 100 by, for example, monitoring the keepalive signaling and/or heartbeats of other controller cores. The Supervisor Controller Core 630 also may receive status messages in the form of electrical keepalive signaling 634 from the Pump Controller Core 620 during infusions, and if something is not as expected, the Supervisor Controller Core 630 may stop the pump motor 600 (by means of stopping power flow via power source 610) if the Pump Controller Core 620 has not already done so. In the event that a controller core is behaving improperly, the Supervisor Controller Core 620 is capable of stopping power flow to the pump motor 600 by means of switching off the power source 610 to the pump motor 600.

In sample configurations, the Supervisor Controller Core 630 monitors electrical keepalive signaling 632 and 634 in the form of pulses at a predetermined frequency from System Controller Core 640 and Pump Controller Core 620, respectively. If any of the keepalive signals 632 or 634 go out of range of a predetermined tolerance (e.g., when the frequency of at least one of the keepalive signaling pulses exceeds an expected frequency range), the Supervisor

Controller Core 630 will deem the relevant controller core(s) is/are behaving improperly. In the event that the Supervisor Controller Core 630 deems a controller core is behaving improperly, it will completely stop pump operation by raising an appropriate alarm and stopping power flow to the pump motor 600 by means of switching off the power source 610 to the pump motor 600.

The System Controller Core 640 and User Interface Controller Core 650 control other operations of the ambulatory peristaltic infusion pump 100 (e.g., graphical user input/output, infusion state logic, alarms, configurations, etc.). These controller cores 640 and 650 are interconnected with each other, the Pump Controller Core 620, and the Supervisor Controller Core 630. Each controller core monitors the operation of the other controller cores as noted above. Also, the Supervisor Controller Core 630 may monitor the other controller cores and detect improper behavior as well.

By separating the functionality of the controller 310 in this manner, operational control safeguards may be maintained whereby the pump motor 600 is immediately inactivated upon detection of improper behavior in any of the controller cores 620-650, thereby minimizing unsafe operation of the ambulatory peristaltic infusion pump 100 described herein as well as other types of infusion pumps known to those skilled in the art.

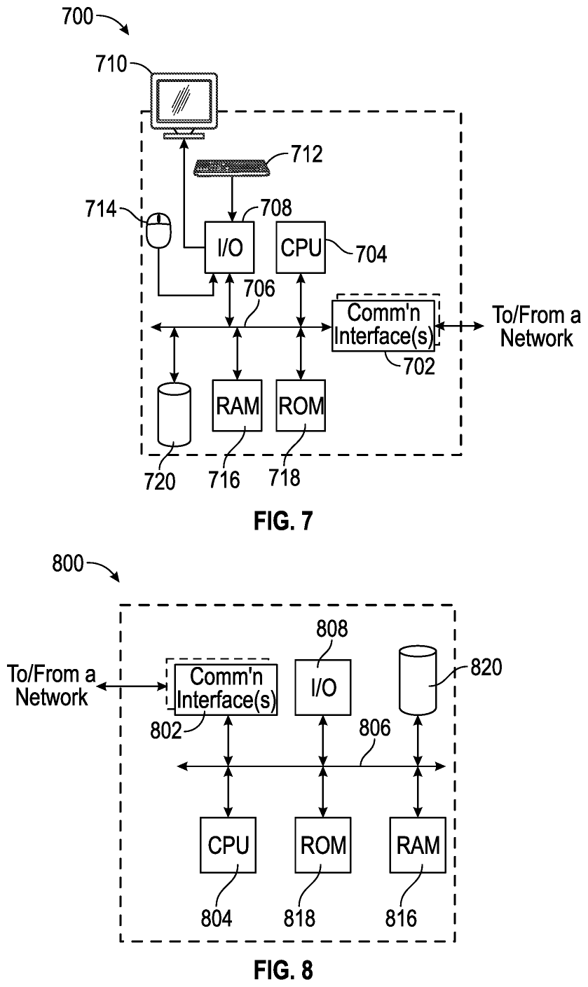

FIGS. 7 and 8 are functional block diagrams illustrating general-purpose computer hardware platforms configured to implement the functional examples described with respect to FIGS. 1-6 as discussed above.

Specifically, FIG. 7 illustrates an example computer platform 700 and FIG. 8 depicts an example computer 800 with user interface elements, as may be used to implement in a personal computer, ambulatory peristaltic infusion pump 100, or other type of workstation or terminal device. It is believed that those skilled in the art are familiar with the structure, programming and general operation of such computer equipment and as a result the drawings should be self-explanatory.

Hardware of an example computer platform 700 (FIG. 7) includes a data communication interface 702 for packet data communication. The computer platform 700 also includes a central processing unit (CPU) 704, in the form of circuitry forming one or more processors, for executing program instructions. The hardware of computer platform 700 typically includes an internal communication bus 706, program and/or data storage 716, 718, and 720 for various programs and data files to be processed and/or communicated by the computer platform 700, although the computer platform 700 often receives programming and data via network communications. In one example, as shown in FIG. 7, the computer platform 700 further includes a video display unit 710 (e.g., a liquid crystal display (LCD) or a cathode ray tube (CRT)), an alphanumeric input device 712 (e.g., a keyboard), a cursor control device 714 (e.g., a mouse), each of which communicate with the internal communication bus 706 via an input/output device (I/O) 708. The hardware elements, operating systems and programming languages of such computer platforms 700 are conventional in nature, and it is presumed that those skilled in the art are adequately familiar therewith. The computer platform 700 may function as a server that may be implemented in a distributed fashion on a number of similar hardware platforms to distribute the processing load.

As illustrated in FIG. 8, hardware of a computer type user terminal device 800, such as a PC (Personal Computer) or tablet computer, similarly includes a data communication interface 802, CPU 804, main memory 816 and 818, one or more mass storage devices 820 for storing user data and the various executable programs, an internal communication bus 806, and an input/output device (I/O) 808.

Aspects of the methods for pump control, as outlined above, may be embodied in programming in general purpose computer hardware platforms (such as described above with respect to FIGS. 7 and 8), e.g., in the form of software, firmware, or microcode executable by a networked computer system such as a server or gateway, and/or a programmable nodal device. Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software, from one computer or processor into another, for example, from a processor 704 of the system 700 and/or from a controller 310 of a peristaltic infusion pump 100 to a computer or software of another system (not shown). Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to one or more of “non-transitory,” “tangible” or “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-transitory storage media include, for example, optical or magnetic disks, such as any of the storage devices in any computer(s) or the like. It may also include storage media such as dynamic memory, for example, the main memory of a machine or computer platform. Tangible transmission media include coaxial cables, copper wire and fiber optics, including the wires that include a bus within a computer system. Carrier-wave transmission media can take the form of electric or electromagnetic signals or acoustic or light waves such as those generated during radio frequency (RF) and light-based data communications. Common forms of computer-readable media therefore include, for example: a floppy disk, a flexible disk, hard disk, magnetic tape, any other magnetic medium, a CD-ROM (compact disc read only memory), DVD (Digital Video Disks) or DVD-ROM, any other optical medium, punch cards paper tape, any other physical storage medium with patterns of holes, a RAM (Random Access Memory), a PROM (Programmable Read Only Memory) and EPROM (Electrically Programmable Read Only Memory), a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

Program instructions may include a software or firmware implementation encoded in any desired language. Programming instructions, when embodied in machine readable medium accessible to a processor of a computer system or device, render the computer system or device into a special-purpose machine that is customized to perform the operations specified in the program performed by the controller 310 of the pump 100.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is ordinary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 105 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that includes a list of elements does not include only those elements but may include other elements not expressly listed or inherent to such process, method, article, or apparatus. An element preceded by “a” or “an” does not, without further constraints, preclude the existence of additional identical elements in the process, method, article, or apparatus that includes the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing describes what is considered to be the best mode and other examples, it is understood that various modifications may be made and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. An infusion pump for pumping fluid through a tube, the infusion pump comprising:
a pump motor configured to pump a fluid through the tube;
a power source for the pump motor; and
a controller for controlling the pump motor, the controller comprising multiple controller cores adapted to ensure proper operation of the pump motor, wherein each controller core controls specific operations of the infusion pump and is interconnected with each other controller core to monitor operation of the other controller cores, the multiple controller cores including a supervisor controller core that is adapted to stop power flow to the pump motor from the power source if any one of the other multiple controller cores is determined to be behaving improperly.

2. The infusion pump of claim 1, wherein the supervisor controller core monitors the health of all other controller cores of the multiple controller cores in the infusion pump.

3. The infusion pump of claim 2, wherein the supervisor controller core monitors the health of other controller cores of the multiple controller cores in the infusion pump by monitoring electrical keepalive pulses at a predetermined frequency and determines that a controller core is behaving improperly when a keepalive signal has a frequency outside of a predetermined tolerance.

4. The infusion pump of claim 1, wherein the multiple controller cores include a pump controller core that controls movement of the pump motor.

5. The infusion pump of claim 1, wherein the multiple controller cores include a system controller core and a user interface controller core that control operations of the infusion pump including at least one of graphical user input/output, infusion state logic, alarms, or configurations.

6. A method of providing control safeguards for an infusion pump including a pump motor configured to pump a fluid through a tube, a power source for the pump motor, and a controller for controlling the pump motor, the controller comprising multiple controller cores adapted to ensure proper operation of the pump motor, each controller core controlling specific operations of the infusion pump and being interconnected with each other controller core, comprising:
each controller core monitoring operation of the other controller cores of the multiple controller cores; and
a supervisor controller core stopping power flow to the pump motor from the power source if any one of the other multiple controller cores is determined to be behaving improperly.

7. The method of claim 6, further comprising the supervisor controller core monitoring the health of all other controller cores of the multiple controller cores in the infusion pump.

8. The method of claim 7, wherein monitoring the health of other controller cores of the multiple controller cores in the infusion pump comprises monitoring electrical keepalive pulses at a predetermined frequency and determining that a controller core is behaving improperly when a keepalive signal has a frequency outside of a predetermined tolerance.

9. The method of claim 6, further comprising controlling movement of the pump motor using a pump controller core of the multiple controller cores.

10. The method of claim 6, further comprising controlling operations of the infusion pump including at least one of graphical user input/output, infusion state logic, alarms, or configurations using at least one of a system controller core or a user interface controller core of the multiple controller cores.

11. A non-transitory computer readable medium including instructions for providing control safeguards for an infusion pump including a pump motor configured to pump a fluid through a tube, a power source for the pump motor, and a controller for controlling the pump motor, the controller comprising multiple controller cores adapted to ensure proper operation of the pump motor, each controller core controlling specific operations of the infusion pump and being interconnected with each other controller core, the instuctions, when executed by the multiple controller cores, performing the following:
each controller core monitoring operation of the other controller cores of the multiple controller cores; and
a supervisor controller core stopping power flow to the pump motor from the power source if any one of the other multiple controller cores is determined to be behaving improperly.
